(19) United States
(12) Patent Application Publication
(10) Pub. No.: US 2006/0110431 A1
(43) Pub. Date: May 25, 2006

(54) Sheets having alternating areas of relatively high and low lotion add-on in the machine direction

(75) Inventors: Kathryn Jean Sirovatka, Neenah, WI (US); Jill Anderson Georger, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/993,560

(22) Filed: Nov. 19, 2004

(51) Int. Cl. A61K 9/70

(52) U.S. Cl. 424/443

(57) ABSTRACT

Lotion-containing sheets, such as tissue sheets or non-woven sheets, having alternating areas of high and low lotion add-on in the machine direction of the sheet have been found to reduce or eliminate downstream build-up of lotion on process equipment.

### BACKGROUND OF THE INVENTION

In the manufacture of tissue products containing lotion, the lotion is generally applied to the surface of the tissue by spraying or printing. On a macro scale, the pattern of the lotion is either uniform or striped in the machine direction of the tissue sheet. In producing facial tissue, for example, in which the tissue sheets are converted on a multi-folder into interfolded clips of tissues for subsequent packaging, a common problem is build-up of the lotion on downstream manufacturing equipment, such as idler rolls and the 45° bars and folding boards on the multi-folder. If lotion build-up becomes excessive, it can cause slits to break on the multi-folder, requiring downtime to rethread the machine.

Therefore there is a need for a means of reducing or eliminating the build-up of lotion on downstream equipment.

### SUMMARY OF THE INVENTION

It has now been discovered that the build-up of lotion on the downstream equipment can be reduced or eliminated by altering the lotion pattern on the tissue such that the downstream equipment “sees” alternating areas of relatively high lotion add-on and relatively low lotion add-on. By so doing, any build-up of lotion on the equipment imparted by a relatively high add-on area is believed to be wiped off by the following low add-on area.

Hence in one aspect, the invention resides in a sheet, such as a non-woven sheet or a tissue sheet, having a machine direction and a cross-machine direction, said sheet containing a patterned surface application of a lotion, said pattern comprising areas of a relatively high add-on amount of lotion and areas of a relatively low add-on amount of lotion which alternately repeat along a line in the machine direction, said areas of a relatively low add-on amount of lotion having a machine direction dimension of about 0.10 inch or greater and said areas of relatively high add-on amount of lotion having a machine direction dimension which is about 300 percent or less of the machine direction dimension of the areas of relatively low add-on amount of lotion.

As used herein, a “sheet” is a non-woven sheet or a tissue sheet that is useful as a wipe, applicator substrate, facial tissue, bath tissue, paper towel and the like. Non-woven sheets are well known in the art and include, without limitation, meltbown, spunbond and coform sheets. Such non-woven sheets are useful for a variety of absorbent personal care product components, wipes, and applicator substrates for applying ointments and the like to the skin. Of particular interest are tissue sheets, which for purposes herein are low density paper sheets made of papermaking fibers and useful for facial tissue or bath tissue and the like. Such sheets are commercially made on high speed tissue machines and therefore inherently have readily apparent “machine direction” and “cross-machine direction” as evidenced by the fiber orientation, directional strength properties and product form characteristics such as slits, folds and perforations.

As used herein, a “patterned” surface application of lotion is a macro pattern of lotion-containing areas such as stripes, squares, dots and the like. The pattern can be regular or irregular. In the case of stripes, the angle of the stripes relative to the machine direction of the sheet can be from about 10 to about 170 degrees, more specifically from about 30 to about 60 degrees and still more specifically from about 40 to about 50 degrees. The shapes, dimensions and add-on amounts of the lotion-containing areas can be the same or different and can vary as viewed in the machine direction. The patterned surface applications of lotion for purposes of this invention comprise alternating areas of a relatively high lotion add-on amount and a relatively low lotion add-on amount, where the dimensions of these regions are discernable to the naked eye. Such macro-patterned surface applications of lotion in accordance with this invention are to be distinguished from “micro” patterns, such as are applied by rotogravure printing, which have alternating micro areas of lotion and no lotion that are not discernable to the naked eye.

As used herein, a “machine direction dimension” is a distance taken along an imaginary line running in the machine direction of the sheet. The patterns useful for purposes of this invention “repeat” in the sense that traversing the surface of the sheet in the machine direction, the lotion add-on amount varies on a macro scale. The repeat can be regular or irregular.

The areas of “relatively high lotion add-on” and “relatively low lotion add-on” may comprise areas of two distinct, single add-on levels, or the “relatively high” areas may comprise a range of one or more add-on levels, all above the mean add-on level on the sheet, while the “relatively low” areas comprise a range of one or more add-on levels, all below the mean add-on level. For convenience, we refer to the mean add-on level within such a “relatively high” add-on range as a single “relatively high” add-on, and similarly, we refer to the mean add-on level within such a “relatively low” add-on range as a single “relatively low” add-on. It is understood that these two terms may refer to single add-on levels or composite add-on levels as just-described. It is possible that the add-on level on the sheet may vary continuously from a maximum level to a minimum level. By the definitions above, the “relatively high” add-on regions would contain the regions with add-ons between the maximum and the mean add-on, while the “relatively low” add-on regions would contain the regions with add-ons between the minimum and the mean add-on.

The lotion add-on amount for the areas of relatively high lotion add-on can be any amount sufficient to provide a benefit to the user. More specifically, the add-on amount can be about 10 grams per square inch (gsm) or less, more specifically from about 1 to about 8 gsm and still more specifically from about 1 to about 5 gsm. The lotion add-on amount for the areas of relatively low lotion add-on can be from 0 to about 80 percent of the add-on amount for the areas of relatively high lotion add-on, more specifically from 0 to about 50 percent and still more specifically from 0 to about 25 percent. Alternatively, the lotion add-on amount for the areas of relatively low lotion add-on can be from 1 to about 80 percent of the add-on amount for the areas of relatively high lotion add-on, more specifically from 1 to about 50 percent and still more specifically from 1 to about 25 percent.

The specific add-on amount of the lotion in the relatively low add-on areas will depend on the amount of lotion in the relatively high add-on amount areas and the relative dimensions of the two areas as measured in the machine direction. Since the relatively low add-on areas are believed to “wipe off” the downstream equipment lotion build-up deposited by the relatively high add-on amount areas, there is flexibility in choosing the optimal combination of pattern sizes and relative add-on amounts. With regard to the size of the areas of relatively low add-on amounts, it is believed that the areas having a relatively low add-on amount of lotion must have a machine direction dimension of about 0.10 inch or greater, more specifically from about 0.25 inch to about 100 inches, more specifically from about 0.25 inch to about 30 inches and still more specifically from about 0.25 inch to about 1 inch. The higher dimensions can arise when stripes of lotion are applied to a sheet at a very small angle relative to the machine direction of the sheet. For other types of patterns or purely cross-machine direction stripes, for example, the lower dimensions would be more likely for a commercial application.

In order to further obtain the benefits of the invention without sacrificing the benefits of lotion add-on, the machine direction dimension of a relatively high lotion add-on amount area must be about 300 percent or less of the machine direction dimension of an adjacent relatively low add-on amount area, more specifically from about 50 to about 300 percent, more specifically from about 100 to about 300 percent and still more specifically from about 100 to about 200 percent of the machine direction dimension of an adjacent relatively low add-on amount area.

The patterned application of lotion can be applied to the surface of the sheet by various means well known in the art, including spraying and printing. Gravure printing is particularly advantageous because of its design flexibility, precision and manufacturing environmental advantages. Suitable rotogravure printing micro patterns provide lotion in uniform surface deposits ranging from about 100 to 1000 deposits per lineal inch. When printing is used to form the patterned application of lotion, the areas of relatively high lotion add-on amount (and the areas of relatively low add-on amount if lotion is present) comprise a micro pattern of many very small deposits.

Lotions useful for purposes of this invention can be any substance that is applied to the surface of a tissue for purposes of improving the feel characteristics of the final product or for delivering a benefit to the user. Such materials are well known in the art. By way of example, without limitation, suitable lotion compositions and their methods of application are disclosed in U.S. Pat. No. 5,665,426 entitled “Soft Treated Tissue” and issued Sep. 9, 1997 to Krzysik et al., which is herein incorporated by reference.

### BRIEF DESCRIPTION OF THE DRAWING

FIG. 1 is a schematic illustration of a prior art tissue sheet having a patterned application of lotion that is susceptible to lotion build-up in downstream equipment.

FIGS. 2-6 are schematic illustrations of sheets in accordance with this invention.

### DETAILED DESCRIPTION OF THE DRAWING

As used in the various figures, like reference numbers represent like features.

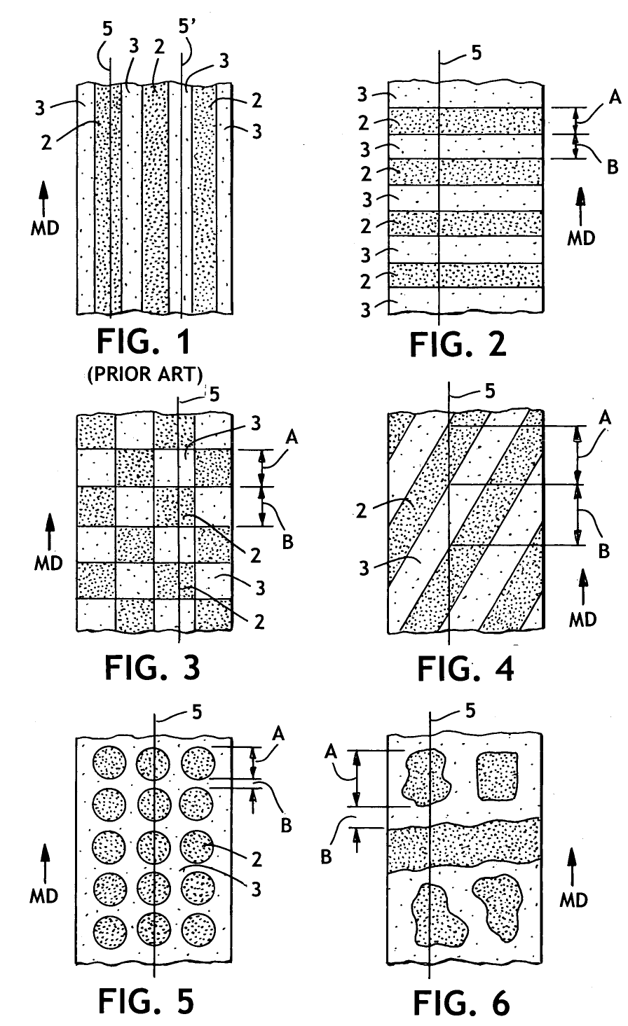

Referring to FIG. 1, shown is a schematic section of a prior art tissue sheet having a plurality of relatively high lotion add-on areas 2 (stripes) running in the machine direction of the sheet. The relatively low lotion add-on areas 3 adjacent the relatively high add-on lotion stripes may contain a lesser add-on amount of lotion or no lotion at all. Imaginary lines 5 and 5′ are drawn to show that there are no areas with the sheet where relatively high add-on areas and relatively low add-on areas alternately repeat along a line in the machine direction, regardless of where the imaginary line is drawn.

FIG. 2 is a schematic section of a sheet in accordance with this invention having a plurality of relatively high lotion add-on areas 2 (stripes) running in the cross-machine direction of the sheet which are separated by relatively low add-on amount areas 3, which are also cross-machine directional stripes. As shown, imaginary line 5 drawn in the machine direction alternately crosses areas of relatively high add-on amount and areas of relatively low add-on amount. The dimension of the relatively high add-on amount in the machine direction is distance “A”. Similarly, the dimension of the relatively low add-on amount in the machine direction is distance “B”. For purposes of this embodiment and all of the embodiments of this invention illustrated below, B≧0.10 inch and, at the same time, A≦3B.

FIG. 3 is a schematic section of a different embodiment of a sheet in accordance with this invention wherein the areas of relatively high add-on and the areas of relatively low add-on are squares or rectangles. As in FIG. 2, imaginary line 5 drawn in the machine direction of the sheet illustrates that the areas of relatively high add-on and the areas of relatively low add-on repeatedly alternate in the machine direction.

FIG. 4 is a schematic section of a different embodiment of a sheet in accordance with this invention wherein the areas of relatively high add-on and the areas of relatively low add-on are diagonal stripes. This embodiment is similar to that of FIG. 2, except the dimensions “A” and “B” are larger than the width of the stripes because of the diagonal orientation.

FIG. 5 is a schematic section of a different embodiment of a sheet in accordance with this invention, wherein the areas of relatively high add-on are circles and the areas of relatively low add-on are the spaces in between.

FIG. 6 is a schematic section of another embodiment of a sheet in accordance with this invention in which the areas of relatively high add-on and relatively low add-on are irregular in shape. As shown, the relatively high add-on areas and relatively low add-on areas alternately repeat along imaginary line 5, albeit that the repeat is irregular. For purposes of this invention, it is not necessary that all add-on areas within the sheet meet the foregoing numerical relationship, as long as there is at least one instance where the relationship applies. Nevertheless, it is preferable that the numerical relationship hold for all areas of the sheet in order to maximize the wiping effect of the relatively low add-on areas.

### EXAMPLES

### Example 1

Tissue sheets were made in accordance with this invention using diagonal stripes as shown in FIG. 4 and the lotion build-up was measured and compared to the lotion build-up when making a tissue sheet using the prior art machine direction stripes of FIG. 1. More specifically, the pattern of relatively high and relatively low add-on regions was obtained by printing the tissue using a gravure roll engraved with alternating, abutting stripes. The relatively high add-on area was 0.75 inch wide (measured perpendicular to the stripe direction) and the engraved cell volume was 5.0 billion cubic microns per square inch (bcm). The relatively low add-on area was 0.406 inch wide and the engraved cell volume was 1.5 bcm. The lotion add-on amount for the relatively high add-on areas was about 3.0 gsm and the lotion add-on amount for the relatively low add-on areas was about 0.5 gsm. For the tissue sheet using diagonal stripes as shown in FIG. 4, the stripes were oriented at a 45° angle. Therefore, the machine direction dimension of the relatively high add-on area was 1.06 inch and the machine direction dimension of the relatively low add-on area was 0.58 inch. Tissues were treated with a lotion containing the following major components: mineral oil (60%); stearyl alcohol (18%); ceresine wax (18%); isopropyl palmitate (3%); and dimethicone (1%).

Approximately 5000 linear yards of tissue were printed with each of the two gravure rolls referenced above. Afterwards, the amount of lotion and fiber built up on a specific downstream idler roll was measured by scraping it off and weighing it. Under otherwise identical conditions, production of the tissue using the machine direction stripe pattern of FIG. 1 (prior art) left 0.21 milligram of lotion and fiber buildup per yard of tissue, while production of tissue in accordance with this invention using the diagonal stripe pattern of FIG. 4 left only 0.08 milligram of buildup per yard.

### Example 2

7500 linear yards of the tissue prepared in Example 1 were rewound over stationary elements intended to mimic a folding board and turn bar on a multifolder. The tissue having machine direction stripes left about half a gram of measurable buildup behind on the stationary elements. No measurable buildup was left behind by the tissue having diagonal stripes in accordance with this invention.

### Example 3

Tissue sheets were made with a diagonal stripe pattern as described in Example 1, except the dimensions of the stripes were halved. That is, the relatively high add-on area was 0.375 inch wide (measured perpendicular to the stripe direction) and the engraved cell volume was 5.0 billion cubic microns per square inch (bcm). The relatively low add-on area was 0.203 inch wide and the engraved cell volume was 1.5 bcm. In accordance with the schematic shown in FIG. 4, the stripes were oriented at a 45° angle. Therefore, the machine direction dimension of the relatively high add-on area was 0.53 inch and the machine direction dimension of the relatively low add-on area was 0.29 inch. Buildup was monitored visually during production of tissues treated with the lotion described in Example 1, and the buildup of lotion and fiber was noted to be less than has been historically observed during production of tissues in accordance with prior art.

It will be appreciated that the foregoing examples, given for purposes of illustration, are not to be construed as limiting the scope of this invention, which is defined by the following claims and all equivalents thereto.

We claim:

1. A sheet having a machine direction and a cross-machine direction, said sheet containing a patterned surface application of a lotion, said pattern comprising areas of a relatively high add-on amount of lotion and areas of a relatively low add-on amount of lotion which alternately repeat along a line in the machine direction, said areas of a relatively low add-on amount of lotion having a machine direction dimension of about 0.10 inch or greater and said areas of relatively high add-on amount of lotion having a machine direction dimension which is about 300 percent or less of the machine direction dimension of the areas of relatively low add-on amount of lotion.

2. The sheet of claim 1 wherein the machine direction dimension of the areas of relatively high add-on amount of lotion is from about 50 to about 300 percent of the machine direction dimension of the areas of relatively low add-on amount of lotion.

3. The sheet of claim 1 wherein the machine direction dimension of the areas of relatively high add-on amount of lotion is from about 100 to about 300 percent of the machine direction dimension of the areas of relatively low add-on amount of lotion.

4. The sheet of claim 1 wherein the machine direction dimension of the areas of relatively high add-on amount of lotion is from about 100 to about 200 percent of the machine direction dimension of the areas of relatively low add-on amount of lotion.

5. The sheet of claim 1 wherein the machine direction dimension of the areas of relatively low add-on amount of lotion is from about 0.25 inch to about 100 inches.

6. The sheet of claim 1 wherein the machine direction dimension of the areas of relatively low add-on amount of lotion is from about 0.25 inch to about 30 inches.

7. The sheet of claim 1 wherein the machine direction dimension of the areas of relatively low add-on amount of lotion is from about 0.25 inch to about 1 inch.

8. The sheet of claim 1 wherein the add-on amount of lotion in the areas of relatively low add-on amount of lotion is from 0 to about 80 percent of the amount of lotion in the areas of relatively high add-on amount of lotion.

9. The sheet of claim 1 wherein the add-on amount of lotion in the areas of relatively low add-on amount of lotion is from about 1 to about 80 percent of the amount of lotion in the areas of relatively high add-on amount of lotion.

10. The sheet of claim 1 wherein the add-on amount of lotion in the areas of relatively low add-on amount of lotion is from 0 to about 50 percent of the amount of lotion in the areas of relatively high add-on amount of lotion.

11. The sheet of claim 1 wherein the add-on amount of lotion in the areas of relatively low add-on amount of lotion is from 0 to about 25 percent of the amount of lotion in the areas of relatively high add-on amount of lotion.

12. The sheet of claim 1 wherein the areas of relatively high add-on amount of lotion comprise from about 100 to about 1000 lotion deposits per lineal inch.

13. The sheet of claim 1 wherein the lotion add-on amount for the areas of relatively high add-on amount lotion is about 10 grams per square inch or less.

14. The sheet of claim 1 wherein the areas of relatively high add-on amount of lotion are stripes.

15. The sheet of claim 1 wherein the areas of relatively high add-on amount of lotion are stripes running at an angle of from about 10 to about 170 degrees relative to the machine direction of the tissue sheet.

16. The sheet of claim 1 wherein the areas of relatively high add-on amount of lotion are stripes running at an angle of from about 30 to about 60 degrees relative to the machine direction of the tissue sheet.

17. The sheet of claim 1 wherein the areas of relatively high add-on amount of lotion are stripes running at an angle of from about 40 to about 50 degrees relative to the machine direction of the tissue sheet.

18. The sheet of claim 1 wherein the areas of relatively high add-on amount of lotion are rectangular.

19. The sheet of claim 1 wherein the areas of relatively high add-on amount of lotion are circular.

20. The sheet of claim 1 wherein the areas of relatively high add-on amount of lotion are irregular in shape.

21. The sheet of claim 1 wherein the sheet is a tissue sheet.

22. The sheet of claim 1 wherein the sheet is a non-woven sheet.
